(19) United States
(12) Patent Application Publication
(10) Pub. No.: US 2024/0226340 A1
(43) Pub. Date: Jul. 11, 2024

(54) METHOD FOR DETERMINING WHETHER AN IMMUNE RESPONSE HAS OCCURRED IN SUBJECTS WHO HAVE BEEN INFECTED WITH- OR VACCINATED AGAINST CORONAVIRUS

(71) Applicant: Dermocovid LLC, Wilmington, DE (US)

(72) Inventors: Julián Alberto Serrano, Santiago del Estero (AR); Maria Amparo Assis Duart, Madrid (ES)

(21) Appl. No.: 18/269,243

(22) PCT Filed: Dec. 23, 2021
(86) PCT No.: PCT/EP2021/087460

§ 371 Date: Jun. 22, 2023

(30) Foreign Application Priority Data
Dec. 23, 2020 (EP) 20383155.7

(51) Int. Cl. A61K 49/00; C07K 14/005

(52) U.S. Cl. CPC A61K 49/0006; C07K 14/005; C12N 2770/20022

(57) ABSTRACT

The present invention refers to the medical field. Particularly, it refers to a method for determining whether an immune response has occurred in subjects who have been infected with- or vaccinated against coronavirus.

### FIELD OF THE INVENTION

The present invention refers to the medical field. Particularly, it refers to a method for determining whether an immune response has occurred in subjects who have been infected with- or vaccinated against coronavirus.

### PRIOR ART

A week after alerting the WHO of a cluster of pneumonia of unknown etiology in Wuhan, the Chinese authorities announced on 7 Jan. 2020 that a novel Coronavirus was identified as the cause of these pneumonia. According to phylogenetic analysis, this novel severe acute respiratory syndrome Coronavirus 2 (SARS-CoV-2), previously named 2019-nCoV, belongs to the B lineage of Betacoronavirus genus and the Sarbecovirus subgenus and has more than 85% nucleotide sequence identity with a bat SARS-like CoV genome published previously. On January 30, WHO declared coronavirus disease-19 (COVID-19) outbreak a public health emergency of international concern and the disease has now spread worldwide.

The COVID-19, currently in pandemic status, caused by SARS-CoV-2, has spread rapidly around the world since the first cases were reported in late 2019. Currently, the number of people who have already overcome the infection is around 78 million worldwide and it is possible that a significant number of people who had the infection asymptomatically were not captured by the corresponding health systems.

One of the strategies to detect all those people who have developed immunity against the virus, after having overcome the infection, is the evaluation of serum antibodies levels against SARS-CoV-2 proteins, particularly protein S or Spike. Thus, tests that detect this antibody response (known as humoral immunity) have been rapidly developed. The information provided by the follow-up of people who have developed immunity helps to estimate the burden of disease, the dynamics of transmission, and the modelling of the epidemic.

However, based on the results reported in different studies, it has been possible to verify that the humoral response, both of IgM and IgG, shows a peak and subsequently declines, without remaining at the long term expected levels. Thus, following 4 to 10 weeks after the disappearance of symptoms, the levels of antibodies show a rapid reduction of the IgG values by half. This evidence raises serious concerns about the robustness and sustainability of the humoral immune response in the post-recovery period, which had been considered crucial for immunity strategy and vaccine development.

Additionally, some people were reported with PCR (polymerase chain reaction) positive for COVID-19 and undetectable levels of protective IgG antibodies, or neutralizing antibodies at very low titters.

On the other side, the emergency of developing vaccination strategies for a disease that is not fully understood has revealed the risk of vaccinating individuals with a high risk of atopy allergy. Huge numbers of people suffer from allergic responses, which can vary in significance from being mildly inconvenient to resulting in rapid death. Allergic responses due to type I hypersensitivity are often referred to as immediate hypersensitivity or atopic allergy and are mediated by the IgE class of antibody bound to tissue mast cells and to circulating blood basophils. Cross-linking of the IgE by the antigen (called allergen in this case) results in the release of a plethora of inflammatory mediators that cause the symptoms. If the allergen reaches the blood stream, the potential death risk increases. Although severe hypersensitivity events are rare, since many countries in the world now recommend and enforce mandatory vaccinations to improve COVID-19 vaccination coverage, the number of hypersensitivity reactions to these vaccines may show an increase. The confirmation of an IgE-mediated response implies recommending that the subject should not be vaccinated.

Thus, in one sense, there is an unmet medical need of finding cost-effective methods to ascertain whether subjects who have been infected with-, or have been vaccinated against Coronavirus, are eliciting an appropriate immune response, for instance a cellular or humoral immune response. On the other, no current methods can detect vulnerable subjects to developing allergic reactions to Coronavirus vaccines. These cost-effective methods could be used for detecting subjects who have already overcome the infection, for evaluating the efficacy of new vaccines, for establishing a priority order among the candidates to receive vaccination, for determining prognostic value of the cellular immune response during the infection, or for assessing possible severe type I hypersensitivity reaction to the vaccine before recommending whether a specific subject should be vaccinated.

The present invention is focused on solving the above cited problem, and a method for identifying immune response in subjects who have been infected with- or vaccinated against Coronavirus is herein described. Kindly note that we are not aware of skin tests for assessing immune response in the context of respiratory viral infections. Thus, the finding of reactive intradermal reaction (IDR) for the SARS-CoV-2 virus would be, to our knowledge, the first case that uses this technique to evaluate an immune response to a respiratory viral infection. Furthermore, our technique offers the advantage of also being able to detect individuals with type I hypersensitivity in whom the vaccine is potentially contraindicated.

### DESCRIPTION OF THE INVENTION

### Brief Description of the Invention

As explained above, the present invention is directed to a method (hereinafter method of the invention) for determining whether an immune response has occurred in subjects who have been infected with- or vaccinated against Coronavirus.

The method of the invention is a cost-effective procedure which could be used in different scenarios, comprising point-of-care testing. The information provided by the method of the invention regarding whether the subjects are eliciting an immune response against Coronavirus insults could be used for different purposes, for instance: for detecting subjects who have already overcome the infection, for evaluating the efficacy of new vaccines, for establishing a priority order among the candidates to receive vaccination, for determining prognostic value of the cellular immune response during the infection or for assessing possible severe type I hypersensitivity reaction to the vaccine before recommending whether a specific subject should be vaccinated.

So, the first embodiment of the present invention refers to Coronavirus-derived antigen/s for use in a method for determining whether an immune response has occurred in subjects who have been infected with- or vaccinated against Coronavirus which comprises: a) Determining the presence of a wheal or skin induration once the Coronavirus-derived antigen has been administered intradermally, and b) wherein the presence of a wheal or skin induration is an indication that the subjects have developed immune response against the Coronavirus infection. Otherwise, this embodiment also refers to a method for determining whether an immune response has occurred in subjects who have been infected with- or vaccinated against Coronavirus which comprises: a) Intradermal administration of Coronavirus-derived antigen/s, b) determining the presence of a wheal or skin induration once the Coronavirus-derived antigen has been administered intradermally, and c) wherein the presence of a wheal or skin induration is an indication that the subjects have developed immune response against the Coronavirus infection.

Thus, the present invention can be used for detecting any kind of immune response which may occur once the subjects have been infected with- or vaccinated against Coronavirus, for instance (non-exhaustive list): Humoral immune response mediated by antibody molecules, preferably IgE-mediated immune response, or cellular immune response mainly mediated by T cells (cell-mediated immunity).

In a preferred embodiment the present invention refers to Coronavirus-derived antigen/s for use in a method for determining whether an IgE-mediated immune response has occurred in subjects who have been infected with- or vaccinated against Coronavirus which comprises: a) Determining the presence of a wheal once the Coronavirus-derived antigen has been administered intradermally, and b) wherein the presence of a wheal between 10-30 minutes following the antigen administration is an indication that the subjects have developed IgE-mediated immune response against the Coronavirus-derived antigen/s. The observation of the wheal at 10-30 minutes after administration evaluates the possible occurrence of IgE-mediated hypersensitivity (also named immediate hypersensitivity, type I hypersensitivity or atopic allergy) to the antigen administered.

In a preferred embodiment the present invention refers to Coronavirus-derived antigen/s for use in a method for recommending whether a subject should be vaccinated against Coronavirus which comprises: a) Determining the presence of a wheal once the Coronavirus-derived antigen has been administered intradermally, and b) wherein the presence of a wheal between 10-30 minutes following the antigen administration is an indication that the subjects have developed IgE-mediated immune response against the Coronavirus-derived antigen/s and the vaccination of these subjects is potentially contraindicated. So, the present invention also refers to a method for the prophylactic treatment of subjects by means of the administration of a vaccine against Coronavirus which comprises a first or previous step of determining whether an IgE-mediated immune response has occurred according to the procedure described above. In this regard, although vaccination would be usually contraindicated when an IgE-mediated immune response has been identified, the subject could be even vaccinated as far as the vaccine is applied by an experienced staff and with specific precautions.

In a preferred embodiment the present invention refers to Coronavirus-derived antigen/s for use in a method for determining whether cell-mediated immune response has occurred in subjects who have been infected with- or vaccinated against Coronavirus which comprises: a) Determining the presence of a skin induration once the Coronavirus-derived antigen has been administered intradermally, and b) wherein the presence of a skin induration between 24-72 hours following the antigen administration is an indication that the subjects have developed cellular immune response against the Coronavirus infection.

In a preferred embodiment the present invention refers to Coronavirus-derived antigen/s for use in a method for determining whether cell-mediated immune response has occurred in subjects who have been infected with- or vaccinated against Coronavirus which comprises: a) Determining the presence of a skin induration once the Coronavirus-derived antigen has been administered intradermally, and b) wherein the presence of a skin induration with a diameter of its major axis, excluding the erythema usually formed around the skin induration, longer than a pre-established threshold value determined in control subjects who have not developed cellular immune response, preferably a diameter of the major axis of at least 4.00 mm, is an indication that the subjects have developed cellular immune response against the Coronavirus infection.

In a preferred embodiment of the present invention, the Coronavirus infection is caused by SARS-CoV-2 and the antigen is a molecule derived thereof which is able to elicit an immune response in the host, comprising (non-exhaustive list): Structural proteins selected from Spike (S), Nucleocapsid (N), Membrane (M) or Envelope (E) protein; inactivated or attenuated viral particles; isolated genetic material or virus-like particles.

In a preferred embodiment of the present invention, the antigen is included in a pharmaceutical composition which comprises an effective amount of the Coronavirus-derived antigen and, optionally, comprises pharmaceutically acceptable excipients and/or carriers. In a preferred embodiment, the doses of Coronavirus derived antigen which is applied to the patient is between 0.050 μg/0.1 mL and 0.500 μg/0.1 mL.

In a preferred embodiment, the Coronavirus-derived antigen is administered by any means able to reach the intradermal space, for example by means of an intradermal injection or a patch test.

In a preferred embodiment the present invention comprises: a) Entering into a computer or device the diameter of the major axis of the wheal or the skin induration, excluding the erythema usually formed around the wheal or skin induration, once the Coronavirus-derived antigen has been administered intradermally; preferably by means of an image taken from the skin, and b) providing a result by the computer or device system regarding the development of immune response based on the information entered according to the step a) and a pre-established threshold value previously stored in the computer, wherein this result is communicated by the computer system to the subject and/or to the authorized health personnel and/or the health system.

The second embodiment of the present invention refers to a method for obtaining information regarding whether an immune response has occurred in subjects who have been infected with- or vaccinated against Coronavirus which comprise performing the steps disclosed above.

The third embodiment of the present invention refers to a data processing apparatus, device or system comprising means for carrying out the method of the invention.

The fourth embodiment of the present invention refers to a computer program product configured for carrying out the method of the invention.

The fifth embodiment of the invention refers to a kit which comprises: a) A medical device, for instance a syringe, for administering intradermally a Coronavirus-derived antigen, and b) instructions for determining the presence of a wheal or skin induration.

In a preferred embodiment the present invention refers to a kit which comprises a) A medical device for administering intradermally a Coronavirus-derived antigen, and b) instructions for determining the presence of a wheal between 10-30 minutes following the antigen administration.

The present invention also refers to the use of this kit for determining whether an IgE-mediated immune response has occurred in subjects who have been infected with- or vaccinated against Coronavirus, or for recommending whether a subject should be vaccinated against Coronavirus.

In a preferred embodiment the present invention refers to a kit which comprises a) A medical device for administering intradermally a Coronavirus-derived antigen, and b) instructions for determining the diameter of the major axis of the skin induration formed between 24-72 hours following the antigen administration. The present invention also refers to the use of this kit for determining whether cell-mediated immune response has occurred in subjects who have been infected with- or vaccinated against Coronavirus.

The sixth embodiment of the present invention refers to SARS-CoV-2-derived antigen for use in a method for determining whether a cellular and/or humoral immune response has occurred in subjects who have been infected with- or vaccinated against SARS-CoV-2 which comprises:
a. Determining the presence of a localized skin immune reaction comprising a wheal skin induration and/or erythema at or around the administration site once the SARS-CoV-2-derived antigen has been administered intradermally,
b. Wherein the presence of a localized skin immune reaction comprising a wheal skin induration and/or erythema at or around the administration site is an indication that the subjects have developed a cellular and/or humoral immune response against the SARS-CoV-2.

In a preferred embodiment of the present invention, the presence of a localized skin immune reaction comprising a wheal, skin induration and/or erythema at or around the administration site with a diameter longer than a pre-established threshold value, is an indication that the subjects have developed cellular and/or humoral immune response against SARS-CoV-2.

In a preferred embodiment of the present invention, the presence of a skin immune reaction comprising a wheal, skin induration and/or erythema at or around the administration site with a diameter of its major axis, longer than a pre-established threshold value is an indication that the subjects have developed a cellular and/or humoral immune response against SARS-CoV-2.

In a preferred embodiment of the present invention, the presence of a skin immune reaction comprising a skin induration and/or erythema at or around the administration site with a diameter of its major axis of a least 4.0 mm, is an indication that the subjects have developed cellular and/or humoral immune response against the SARS-CoV-2.

In a preferred embodiment, the present invention comprises assessing the presence or amount of infiltrating immunological cells (comprising their progenitors, immature, mature, activated and/or exhausted subsets) and/or related markers (including clusters of differentiation [CDs, such as CD4 as CD4⁺ T cells marker, CD8 as CD8⁺ T cells marker and CD20 as B cells marker], cytokines, chemokines, receptors, transporters, enzymes, transcriptions factors, proteins, ligands, miRNAs, metabolites, alarmins and defensins) in a biological sample obtained from the area of wheal, skin induration and/or erythema, wherein the presence of an increased amount of infiltrating immunological cells and/or their related markers with respect to a pre-established threshold value, is an indication that the subjects have developed cellular and/or humoral immune response against the SARS-CoV-2.

In a preferred embodiment, the present invention comprises assessing the presence or amount of infiltrating lymphocytes (including CD4⁺ T cells, CD8⁺ T cells and B cells), other mononuclear cells (including monocytes/macrophages and NK cells), polymorphonuclear cells (neutrophils, eosinophils and basophils), mast cells and/or related markers in a biological sample obtained from the area of wheal, induration and/or erythema, wherein the presence of an increased amount of infiltrating lymphocytes, other mononuclear cells, polymorphonuclear cells, mast cells and/or related markers, with respect to a pre-established threshold value, is an indication that the subjects have developed cellular and/or humoral immune response against the SARS-CoV-2.

In a preferred embodiment, the present invention comprises assessing the presence or amount of infiltrating T cells, other mononuclear cells (including NK cells, monocytes/macrophages and other antigen-presenting cells), polymorphonuclear cells (including neutrophils) and/or related markers (including CD4 and CD8) in a biological sample obtained from the area of induration and/or erythema, wherein the presence of an increased amount of infiltrating T cells, other mononuclear cells, polymorphonuclear cells and/or related markers, with respect to a pre-established threshold value, is an indication that the subjects have developed cellular immune response against the SARS-CoV-2.

In a preferred embodiment, the present invention comprises assessing CD4⁺/CD8⁺ T cells ratio among the infiltrating cells in a biological sample obtained from the area of induration and/or erythema wherein the identification of a CD4⁺/CD8⁺ T-cells ratio different than a pre-established threshold ratio value, is an indication that the subjects have developed cellular immune response against the SARS-CoV-2.

In a preferred embodiment, the present invention comprises assessing CD4⁺/CD8⁺ T cells ratio among the infiltrating cells in a biological sample obtained from the area of induration and/or erythema, wherein the identification of a CD4⁺/CD8⁺ T cells ratio different than 1/1 is an indication that the subjects have developed cellular immune response against the SARS-CoV-2.

In a preferred embodiment, the present invention comprises assessing the presence or amount of infiltrating B cells and/or related markers (including CD20, BCR and immunoglobulins) in a biological sample obtained from the area of induration and/or erythema, wherein the presence of an increased amount of infiltrating B cells and/or their related markers (including CD20, BCR and immunoglobulins) with respect to a pre-established threshold value, is an indication that the subjects have developed humoral immune response against the SARS-CoV-2.

In a preferred embodiment, the present invention comprises assessing the presence or amount of infiltrating innate mononuclear cells (including monocytes/macrophages, NK cells, NKT cells and innate lymphoid cells), polymorphonuclear cells and/or related markers in a biological sample obtained from the area of induration and/or erythema, wherein the presence of an increased amount of infiltrating innate mononuclear cells, polymorphonuclear cells and/or their related markers with respect to a pre-established threshold value, is an indication that the subjects have developed cellular innate immune response against the SARS-CoV-2.

In a preferred embodiment, the present invention comprises assessing the presence or amount of infiltrating eosynophils, basophils, mast cells and/or related markers in a biological sample obtained from the area of wheal, induration and/or erythema, wherein the presence of an increased amount of infiltrating eosynophils, basophils, mast cells and/or their related markers with respect to a pre-established threshold value, is an indication that the subjects have developed IgE-mediated immune response against the SARS-CoV-2.

In a preferred embodiment, the present invention comprises performing an image examination to assess the characteristics of the dermis and subcutaneous cell tissues in the area at or around the antigen administration, wherein the identification of a different imaging pattern (i.e. typically the presence of cellulitis) with respect to a pre-established imaging pattern, is an indication that the subjects have developed cellular and/or humoral immune response against the SARS-CoV-2.

In a preferred embodiment, the present invention comprises performing an image examination to assess the thickness of the dermis and subcutaneous cell tissues in the area at or around the antigen administration, wherein the identification of a thickness value higher than a pre-established threshold thickness value, is an indication that the subjects have developed cellular and/or humoral immune response against the SARS-CoV-2.

In a preferred embodiment, the present invention comprises performing an image examination to assess the thickness of the dermis and subcutaneous cell tissues in the area at or around the antigen administration, wherein the identification of a thickness value of at least twice a pre-established threshold value, preferably a thickness value of at least 2 mm, is an indication that the subjects have developed cellular and/or humoral immune response against the SARS-CoV-2.

In a preferred embodiment, the present invention comprises determining whether an IgE-mediated immune response has occurred in subjects who have been infected with- or vaccinated against SARS-CoV-2 by determining the presence of a wheal in the area at or around where the SARS-CoV-2-derived antigen has been administered intradermally, wherein the presence of a wheal with a diameter higher than a pre-established threshold, between 10-30 minutes following the antigen administration, is an indication that the subjects have developed IgE-mediated immune response against the SARS-CoV-2 and the vaccination of these subjects is potentially contraindicated.

The present invention also refers to SARS-CoV-2-derived antigen for use in a method for recommending whether a subject should be vaccinated against SARS-CoV-2 by determining the presence of a wheal in the area at or around once the SARS-CoV-2-derived antigen has been administered intradermally, wherein the presence of a wheal with a diameter higher than a pre-established threshold, between 10-30 minutes following the antigen administration, is an indication that the subjects have developed IgE-mediated immune response against the SARS-CoV-2 infection and the vaccination of these subjects is potentially contraindicated.

In a preferred embodiment, the present invention comprises determining whether cellular immune response has occurred in subjects who have been infected with- or vaccinated against SARS-CoV-2 by determining the presence of a skin induration and/or erythema at or around once the SARS-CoV-2-derived antigen has been administered intradermally, wherein the presence of a skin induration and/or erythema between 18-72 hours following the antigen administration is an indication that the subjects have developed cell-mediated immune response against the SARS-CoV-2.

In a preferred embodiment, the present invention comprises determining whether IgG and/or IgM-mediated immune response has occurred in subjects who have been infected with- or vaccinated against SARS-CoV-2 by determining the presence of a skin induration and/or erythema at or around once the SARS-CoV-2-derived antigen has been administered intradermally, wherein the presence of a skin induration and/or erythema between 4-24 hours following the antigen administration is an indication that the subjects have developed IgG and/or IgM-mediated immune response against the SARS-CoV-2.

In a preferred embodiment the above cited variables have a diagnostic and/or prognostic value, so they can be used in an in vitro and/or ex vivo method for the diagnosis or identification of recovered unvaccinated patients who have been previously exposed to a SARS-CoV-2 infection, or patients with ongoing SARS-CoV-2 infection; the prognosis of a patient already suffering from a SARS-CoV-2 infection and/or the prognosis of disease severity in vaccinated subjects in case of future SARS-CoV-2 infection.

In a preferred embodiment, the antigen is a molecule derived from SARS-CoV-2 which is able to elicit an immune response in the host, comprising: Structural proteins selected from Spike (S), Nucleocapsid (N), Membrane (M) or Envelope (E) protein or peptides derived from them; inactivated or attenuated viral particles; isolated genetic material or virus-like particles.

In a preferred embodiment, the antigen is included in a pharmaceutical composition which optionally comprises pharmaceutically acceptable excipients and/or carriers.

In a preferred embodiment, the present invention refers to a computer-implemented method which comprises:
a. Receiving by a computer program the variables assessed according to the present invention, once the SARS-CoV-2-derived antigen has been administered intradermally; preferably by means of an image taken from the skin;
b. Process the values according to step a) for finding a statistically significant variation or deviation;
c. Providing a result by the computer system regarding the development of immune response based on the information entered according to the step a) and a pre-established threshold value already stored in the computer, wherein this result is communicated by the computer system to the subject and/or to the authorized health personnel and/or the health system.

The present invention also refers to a data processing apparatus, device or system comprising means for carrying out the above computer-implemented method.

The present invention also refers to a computer program product configured for carrying out the above computer-implemented method.

The present invention also refers to a kit which comprises:
a. A medical device for administering intradermally a SARS-CoV-2-derived antigen, and
b. Instructions for determining the presence of a wheal, skin induration and/or erythema at or around the administration site.

In a preferred embodiment the kit comprises instructions for determining the presence of a wheal at or around the administration site between 10-30 minutes following the antigen administration.

In a preferred embodiment the kit comprises instructions for determining the diameter of the major axis of the skin induration and/or erythema at or around formed between 18-72 hours or 4-24 hours following the antigen administration.

For the purpose of interpreting the present invention the following terms are defined:
The term “comprising” means including, but it is not limited to, whatever follows the word “comprising”. Thus, use of the term “comprising” indicates that the listed elements are required or mandatory, but that other elements are optional and may or may not be present.
By “consisting of” means including, and it is limited to, whatever follows the phrase “consisting of”. Thus, the phrase “consisting of” indicates that the listed elements are required or mandatory, and that no other elements may be present.
By “effective dose or amount” is intended an amount that, when administered as described herein, brings about a measurable immune response in the host. The exact amount required will vary from subject to subject, depending on the age, and general condition of the subject, the severity with which the disease has been/is progressed, mode of administration, and the like. An appropriate “effective” amount in any individual case may be determined by one of ordinary skill in the art using routine experimentation, based upon the information provided herein.
A reference control level is mentioned herein, when referring to any of the variables assessed in the present invention, for example wheal, skin induration and/or erythema diameter, presence or amount of infiltrating immunological cells, image examination, etc. Particularly, a reference control level is mentioned when referring to the presence or amount of infiltrating immunological cells and/or their related markers and the CD4⁺/CD8⁺ T cells ratio among the infiltrating cells in a biological sample obtained from the area of wheal, skin induration and/or erythema, as well as when referring to the imaging pattern and the thickness of the dermis and subcutaneous cell tissues in the area at or around the antigen administration. The subject is likely to elicit a cellular and/or humoral immune response with a given sensitivity and specificity if variable assessed is different, preferably above or below, said reference control level. A reference value can be a “threshold value” or a “cut-off” value. Typically, a “threshold value” or “cut-off value” can be determined experimentally, empirically, or theoretically. According to the present invention, the “pre-established threshold” value refers to a value previously determined in subjects who have not developed cellular nor humoral immune response or in subjects who received i.d. vehicle/control administration. The patient is likely to have an immune response with a given sensitivity and specificity if the variable measured in the patient are different, above or below, said “pre-established threshold level”. A “threshold value” can also be arbitrarily selected based upon the existing experimental and/or clinical conditions, as would be recognized by a person of ordinary skilled in the art. The “threshold value” has to be determined in order to obtain the optimal sensitivity and specificity according to the function of the test and the benefit/risk balance (clinical consequences of false positive and false negative). Typically, the optimal sensitivity and specificity (and so the “threshold value”) can be determined using a Receiver Operating Characteristic (ROC) curve based on experimental data. For example, after determining the levels of the biomarker in a group of reference, one can use algorithmic analysis for the statistic treatment of the measured concentrations of biomarkers in biological samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-speciftcity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve.
By “major axis of the wheal, skin induration and/or erythema” it is understood the greatest distance between two opposite points on the wheal, skin induration and/or erythema which is formed in the area at or around where the coronavirus-derived antigen/s is/are administered to the subject.

### BRIEF DESCRIPTION OF THE FIGURES

FIG. 1: Anti-spike IgG antibodies serum levels from SARS-CoV-2 among COVID-19 and control cases. ****p<0.0001, unpaired Student's t-test.

FIG. 2. IDR measurement after 24 h and 48 h of an i.d. administration of spike protein (S protein) at different doses. The induration induced by the IDR is reported as the diameter of its major axis, without the surrounding erythema. ****p<0.0001, unpaired Student's t-test.

FIG. 3. IDR specific to SARS-CoV-2 virus spike protein (S protein) in 9 convalescent subjects infected with SARS-CoV-2. Of the 9 subjects studied, 7 had mild symptoms while 2 showed severe symptoms, and only 4 were evaluated at two time points. The magnitude of in vivo responses for each individual was evaluated 48 (a) or 24 (b) hours following the i.d. injection. Each coloured segment represents a different dose of protein i.d. injected in 0.1 mL of vehicle.

FIG. 4. CD4⁺/CD8⁺ T cells ratio from papillary dermis infiltrates following i.d. administration of spike protein or vehicle solution. Immunohistochemistry technique was used to evaluate the presence or amount of infiltrating immune cells in biopsies samples from the site of administration, 24 h following spike protein (S protein) or vehicle i.d. injection in subjects that had been vaccinated against or had been infected with SARS-CoV-2. ****p<0.0001, unpaired Student's t test.

FIG. 5. Thickness of dermis and subcutaneous cell tissues in millimeters (mm). Ultrasound image was used as a non-invasive technique to evaluate imaging of the site of administration, 24 h following spike protein (S protein) or vehicle i.d. injection in subjects that had been vaccinated against or had been infected with SARS-CoV-2. ****p<0.0001, unpaired Student's t test.

### DETAILED DESCRIPTION OF THE INVENTION

The present invention is illustrated by means of the Examples set below, without the intention of limiting its scope of protection.

### Assessment of the Presence of a Wheal, Erythema or Skin Induration

### Example 1. Materials and Methods

Humans: Volunteers were recruited based on their clinical history of SARS-CoV-2 infection (diagnosed by positive PCR for the SARS-CoV-2 virus in nasopharyngeal swab). The studies on subjects who recovered from COVID-19 (n=9) were tested after the symptom's onset. Healthy subjects without a clinical history of COVID-19 (n=6) were simultaneously recruited, between October and December 2020. All healthy donor samples tested negative for IgG antibodies to S protein.

Inclusion criteria: Individuals aged 20 to 76 years, of both sexes.

Exclusion criteria: Individuals who suffer infectious diseases, acute or chronic inflammatory diseases, treatment with immunomodulatory drugs (systemic corticosteroids), cancer, kidney failure, heart failure, immunodeficiencies or autoimmune diseases.

Ethical and legal aspects: Volunteers were informed of the purpose and methods of the study in order to obtain their written consent, developed in compliance with the Declaration of Helsinki and the Good Clinical Practice Guidelines.

Reagents and materials: Protein S of the SARS-CoV-2 virus were used, dissolved in a vehicle solution composed of phosphate buffer saline (PBS, pH=7.4) and different conservatives. The vehicle solution was used as a negative control of the reaction. Sterile 1 mL syringes and hypodermic needles were used for intradermal injection (i.d.), as described below.

Computer application: Applications for iPhone and Android cell phones to capture images and an artificial intelligence to analyse images were developed.

Humoral response: On day 1, peripheral blood was obtained by venipuncture previous to the procedure (see below). The samples were centrifuged for 5 minutes at 2000 r.p.m. and the serum separated in order to determine the levels of IgG anti-protein S by chemiluminescence as a parameter for humoral response.

### Example 2. Procedure

Intradermal reaction (IDR): On day 1, 0.1 mL of the solution containing protein S from SARS-CoV-2 were i.d. injected on the anterior surface of the forearm, previously disinfected with a suitable germicide, which were allowed to dry before injection. The presence of a wheal, skin induration and/or erythema in the area at or around the administration site was evaluated 10-30 minutes and 24 hours and 48 hours after the injection (days 2 and 3, respectively). At 10-30 minutes following administration, the presence or absence of a wheal was reported. At 24 hours and 48 hours following administration, the diameter of the major axis of the skin induration (preferably excluding the erythematous area) was measured in millimetres (mm) with a graduated ruler in a double-blind manner. If there were, the presence and size of lesions or edema were also recorded.

Dose-response curve: 3 injections were applied in each subject with different doses of protein, according to the following schedule:
A: 0 μg/0.1 mL
B: 0.145 μg/0.1 mL
C: 0.28 μg/0.1 mL

### Statistical Analysis

For comparison, two tailed Student's t-tests were applied unless otherwise specified. Statistical tests were performed using GraphPad.7 for Windows.

### Example 3. Results

### Clinical Characteristics

Table 1 shows the clinical characteristics of the 9 confirmed cases (56% male, 44% female), who were diagnosed by the Ministry of Public Health of Santiago del Estero, Argentina, between 3 Aug. 2020 and 17 Nov. 2020, with mean 111 (±28) days after onset of symptom, and 6 control cases (50% male, 50% female). In COVID-19 cases, the average age was 41.9 (8.7) years and no subjects older than 60, while in control cases the average age was 49.2 (13.1) years and 1 subject older than 60. All subjects were residents of Santiago del Estero city, Argentina, or surrounding areas. Among COVID-19 cases, 1 (11%) had underlying hypothyroidism and overweight, and 2 (22%) were smokers, while among controls, only 1 (17%) smoker was reported.

TABLE 1

| Subject | | | | | COVID-19 | Days |
|---|---|---|---|---|---|---|
| ID | Group | Age | Gender | Comorbidities | severity | post-PCR |
| Con1 | Control | 46 | F | — | — | — |
| Con2 | Control | 76 | M | — | — | — |
| Con3 | Control | 52 | M | — | — | — |
| Con4 | Control | 42 | M | Smoker | — | — |
| Con5 | Control | 34 | F | — | — | — |
| Con6 | Control | 45 | F | — | — | — |
| Cov1 | COVID-19 | 49 | F | Hypothyroidism | Mild | 96 |
| | | | | Overweight | | |
| Cov2 | COVID-19 | 41 | M | — | Mild | 23 |
| Cov3 | COVID-19 | 34 | F | — | Asymptomatic | 127 |
| Cov4 | COVID-19 | 44 | F | — | Moderate | 129 |
| Cov5 | COVID-19 | 47 | M | Smoker | Mild | 59 |
| Cov6 | COVID-19 | 44 | F | — | Moderate | 128 |
| Cov7 | COVID-19 | 47 | M | Smoker | Mild | 128 |
| Cov8 | COVID-19 | 21 | M | — | Mild | 128 |
| Cov9 | COVID-19 | 50 | M | — | Mild | 82 |

Clinical characteristics of the included subjects

### Anti-Spike IgG Levels in COVID-19 Cases

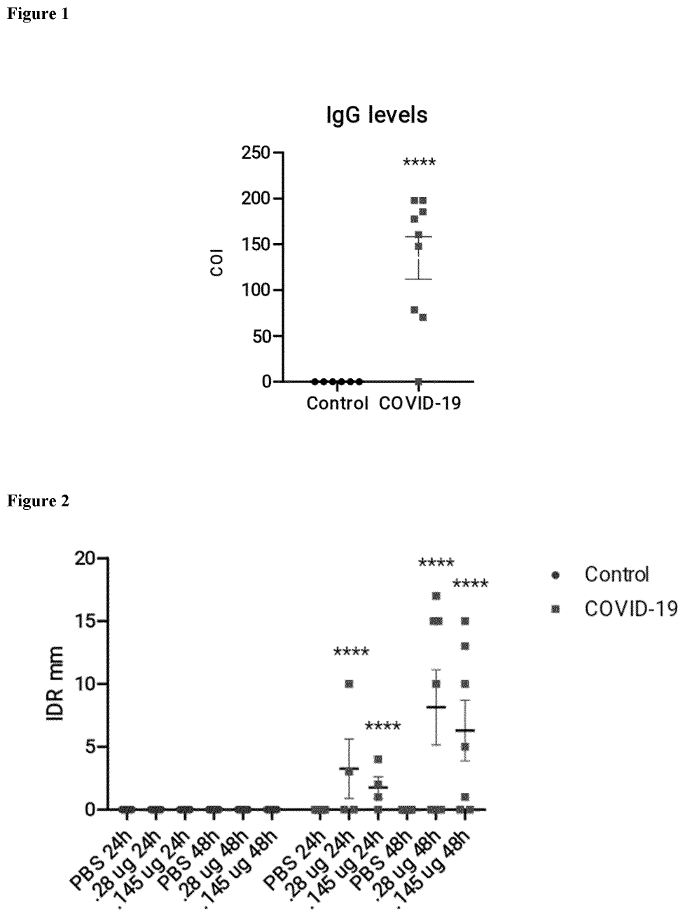

Anti-spike IgG levels detected by chemiluminescence in serum samples obtained from COVID-19 and control cases at different periods after disease onset are shown in Table 2. Results are semi quantitative and are expressed as qualitative statements (reactive/non-reactive). A Cut-off-Index (COI)≥1 is considered reactive. The average IgG levels was higher in COVID-19 group (0.1±0.00 COI) than in their control counterparts (135.2±23.2 COI, FIG. 1). Only 1 (110%) COVID-19 case did not show detectable IgG levels, as were also observed for all the control cases.

TABLE 2

| | IDR 10-30 | | | | | | | | | | | |
|---|---|---|---|---|---|---|---|---|---|---|---|---|
| | min (mm) | | | IDR 24 h (mm) | | | IDR 48 h (mm) | | | IgG | COVID- | Days |
| Subject | 0 | .145 | .28 | 0 | .145 | .28 | 0 | .145 | .28 | levels | 19 | post- |
| ID | μg | μg | μg | μg | μg | μg | μg | μg | μg | (COI) | severity | PCR |
| Cov2 | 0 | 0 | 0 | 0 | 0 | 0 | 0 | 0 | 0 | 0.2 | M | 23 |
| Cov5 | 0 | 0 | 0 | Nd | Nd | Nd | 0 | 10 | 0 | 160.5 | M | 59 |
| Cov9 | 0 | 0 | 0 | Nd | Nd | Nd | 0 | 20 | 0 | 198 | M | 82 |
| Cov1 | 0 | 0 | 0 | 0 | 1 | 10 | 0 | 1 | 15 | 185.6 | M | 96 |
| Cov3 | 0 | 0 | 0 | 0 | 2 | 3 | 0 | 0 | 0 | 70.6 | A | 127 |
| Cov6 | 0 | 0 | 0 | Nd | Nd | Nd | 0 | 13 | 17 | 177.7 | Mo | 128 |
| Cov7 | 0 | 0 | 0 | Nd | Nd | Nd | 0 | 15 | 15 | 147.9 | M | 128 |
| Cov8 | 0 | 0 | 0 | Nd | Nd | Nd | 0 | 0 | 10 | 78.7 | M | 128 |
| Cov4 | 0 | 0 | 0 | 0 | 4 | 0 | 0 | 7 | 6 | 198 | Mo | 129 |
| Con1 | 0 | 0 | 0 | 0 | 0 | 0 | 0 | 0 | 0 | 0.1 | — | — |
| Con2 | 0 | 0 | 0 | 0 | 0 | 0 | 0 | 0 | 0 | 0.1 | — | — |
| Con3 | 0 | 0 | 0 | 0 | 0 | 0 | 0 | 0 | 0 | 0.1 | — | — |
| Con4 | 0 | 0 | 0 | 0 | 0 | 0 | 0 | 0 | 0 | 0.1 | — | — |
| Con5 | 0 | 0 | 0 | 0 | 0 | 0 | 0 | 0 | 0 | 0.1 | — | — |
| Con6 | 0 | 0 | 0 | 0 | 0 | 0 | 0 | 0 | 0 | 0.1 | — | — |

IDR measures and serum IgG levels against Spike protein of SARS-CoV-2. The IDR values are shown in millimetres (mm) following 24 h or 48 h of i.d. injection of different doses of Spike protein. The days before symptoms onset and the severity of the disease are also shown. Cov, COVID-19 case; Con, Control case; Nd, Not determined; M, Mild; A, Asymptomatic; Mo, Moderate.

### Intradermal Reaction (IDR)

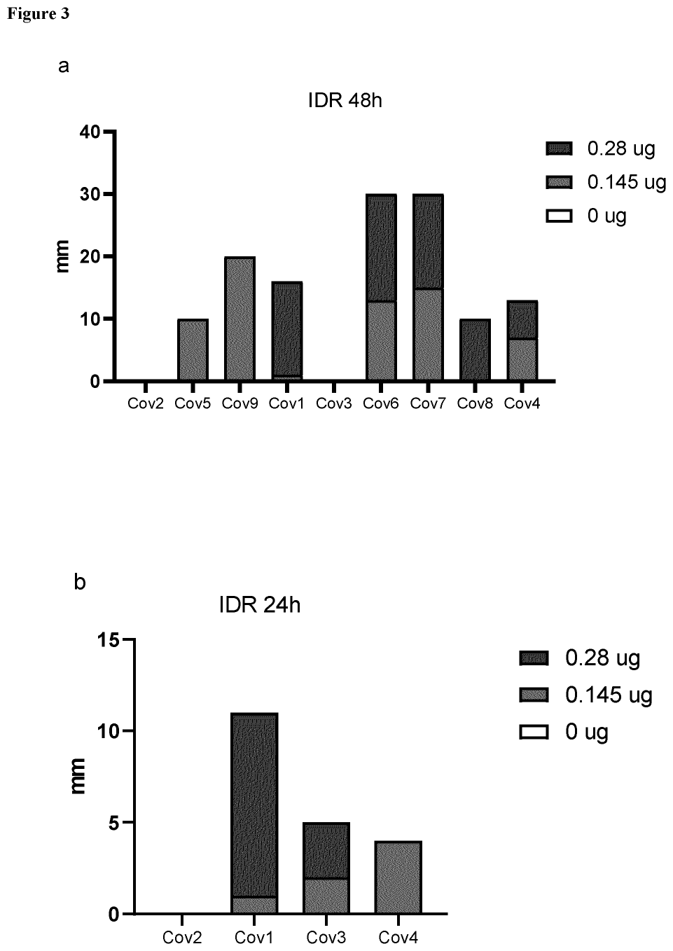

Between 10-30 minutes following injection, no wheals were observed in any case. Mean IDR size at 48 h with 0.145 μg and 0.28 μg of S protein were 7.33 mm and 7.00 mm, respectively, referred as the diameter of the major axis of the skin induration, preferably without erythema. Only 4 COVID-19 cases were also measured at 24 h following injection, and the mean IDR size with 0.145 μg and 0.28 μg of S protein were 7.33 mm and 7.00 mm, respectively (FIG. 2). No differences were observed among COVID-19 and control group about vehicle (0 μg of protein) injection (Table 2 and FIG. 3). No lesions, edema, or unwanted effects were observed in any case.

### Assessment of a Biosignature and/or Imaging Patterns

### Example 4. Material and Methods

Humans: Volunteers were recruited based on their clinical history of SARS-CoV-2 infection (diagnosed by positive PCR for the SARS-CoV-2 virus in nasopharyngeal swab) and/or for having received the complete series of COVID-19 vaccine. The studies on subjects who recovered from COVID-19 were tested after the symptoms onset and, in case of vaccinated subjects, at least one month after the last dose. All the subjects (n=7) were recruited during August 2021 for Immunohistochemistry determinations and during September 2021 for ultrasound determinations.

Inclusion criteria: individuals aged 20 to 76 years, of both sexes.

Exclusion criteria: individuals who suffer infectious diseases, acute or chronic inflammatory diseases, treatment with immunomodulatory drugs (systemic corticosteroids), cancer, kidney failure, heart failure, immunodeficiencies or autoimmune diseases.

Ethical and legal aspects: the volunteers were informed of the purpose and methods of the study in order to obtain their written consent, developed in compliance with the Declaration of Helsinki and the Good Clinical Practice Guidelines.

Reagents and materials: Spike protein (S protein) of the SARS-CoV-2 virus were used, dissolved in a vehicle solution composed of phosphate buffer saline (PBS, pH=7.4) and different conservatives. The vehicle solution were used as a negative control of the reaction. Sterile 1 mL syringes and hypodermic needles were used for intradermal injection (i.d.), as described below.

Intradermal reaction (IDR): On day 1, 0.1 mL of the solution containing S protein from SARS-CoV-2 were i.d. injected on the anterior surface of the left forearm and 0.1 mL of the vehicle solution on the anterior surface of the right forearm, previously disinfected with a suitable germicide, which were allowed to dry before injection. The presence of an induration and/or erythema in the area at or around the administration site was evaluated 24 h after the injection (day 2). The largest diameter of the palpable induration (preferably excluding the erythematous area) was measured in millimeters (mm) with a graduated ruler in a double-blind manner. If there were, the presence and size of lesions or edema were also recorded.

Biopsy technique: Biopsies were taken 24 h post-intradermal challenge. The area to be biopsied was disinfected with a suitable germicide and anaesthetized using subcutaneous (s.c.) 2% Lidocaine. Then, a Sovereing 3 mm sterile punch biopsy was introduced in the area of interest and rotating to obtain a tissue specimen. Biopsies were removed from the middle of the induration for S protein injections or from the middle of the circled area for vehicle control injections. Immediately after collection, the biopsies were fixed in 10% formaldehyde for 24 hs at room temperature for being then embedded in paraffin.

Immunohistochemistry: Cross-sections of paraffin-embedded tissue specimen were analyzed by chromogenic immuno-histochemical staining, to evaluate the presence or amount of infiltrating CD4⁺ and CD8⁺ T cells, CD20⁺ B cells, other mononuclear cells (such as monocytes/macrophages) and polymorphonuclear cells in the skin punch biopsies by a certified pathologist in a double-blind manner.

Ultrasound images of skin, in vivo: A high-frequency ultrasound scanner (Esaote MyLab X8, Italy) equipped with a multifrequency (4-15 MHz) electronic linear array transducer was used as a non-invasive technique in order to find imaging characteristics in the superficial skin with optimal spatial resolution. Ultrasound images with distinguishable skin layers were also analyzed for the overall skin thickness of different layers including the stratum corneum, epidermis, dermis, and hypodermis. Skin layers were distinguished based on their order, appearance, and echogenicity with light structures characterized as hyperechoic, white, or hypoechoic, gray, or darker areas. The thickening of the dermis and subcutaneous cell tissue was measured in millimeters (mm) and the characteristics of the imaging pattern was determined at or around the administration site, 24 h after the i.d. injection of S protein, compared to the i.d. injection of vehicle control solution.

### Statistical Analysis

For comparison, two tailed Student's t-tests were applied unless otherwise specified. Statistical tests were performed using GraphPad.7 for Windows.

### Example 5. Results

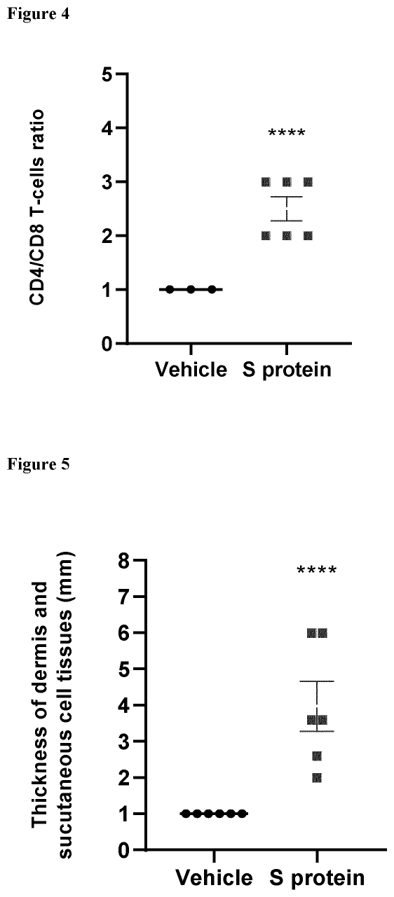

Immunohistochemistry: On histologic examination, the most common pattern of inflammation following spike injection was a mild perivascular infiltrate that was not observed after vehicle injection. In some cases, following spike injection, the infiltrate was also periadnexal and, in one case, a perineural infiltrated was observed too. Following spike administration, the inflammatory infiltrated were predominantly lymphocytic mononuclear cells, with no significant presence of polymorphonuclear leukocytes neither macrophages. No CD20⁺ B cells were mostly seen, except after two cases of spike i.d. administration (see Table 3). In all the cases where spike protein were injected, a significant increase in CD4⁺/CD8⁺ T cells ratio was observed compared to control injections (FIG. 4).

Ultrasound image: The in vivo ultrasound image examination revealed a significant increase in the thickness of the dermis and subcutaneous cell tissues following spike injection compared to vehicle injection (FIG. 5). In addition, in all cases following spike injection, the corresponding imaging pattern demonstrated presence of cellulitis at or around the administration site, which was not observed following vehicle injection.

TABLE 3

| | | | | Ratio | | mm |
|---|---|---|---|---|---|---|
| | | | | CD4/ | Inflammatory | and |
| Condition | CD20 | CD4 | CD8 | CD8 | reaction pattern | IgG |
| Vehicle 1 | Negative | Positive on | Positive on | 1/1 | No significant | 0 |
| | | perivascular | perivascular | | inflammatory | |
| | | mature- | mature- | | infiltrate observed | |
| | | looking T- | looking T- | | | |
| | | cells | cells | | | |
| Vehicle 2 | Negative | Positive on | Positive on | 1/1 | No significant | 0 |
| | | perivascular | perivascular | | inflammatory | |
| | | mature- | mature- | | infiltrate observed | |
| | | looking T- | looking T- | | | |
| | | cells | cells | | | |
| Vehicle 3 | Negative | Positive on | Positive on | 1/1 | No significant | 0 |
| | | perivascular | perivascular | | inflammatory | |
| | | mature- | mature- | | infiltrate observed | |
| | | looking T- | looking T- | | | |
| | | cells | cells | | | |
| Spike 1 | Negative | Positive on | Positive on | 2/1 | Mild perivascular | 4 mm |
| (COVID + | | perivascular | perivascular | | inflammatory | 0 COI |
| 2 doses of | | mature- | mature- | | reaction pattern in | |
| Sputink V | | looking T- | looking T- | | papillary dermis. | |
| vaccine) | | cells | cells | | Inflammatory | |
| | | | | | infiltrate with | |
| | | | | | predominantly | |
| | | | | | mononuclear cells. | |
| | | | | | Macrophages: NS | |
| | | | | | Polymorphonuclear | |
| | | | | | cells: NS | |
| Spike 2 | Positive | Positive on | Positive on | 3/1 | Perivascular | 11 mm |
| (2 doses of | on | perivascular | perivascular | | inflammatory | 34 COI |
| Sinopharm | isolated | mature- | mature- | | reaction pattern in | |
| vaccine) | mature- | looking T- | looking T- | | papillary dermis. | |
| | looking | cells | cells | | Inflammatory | |
| | B-cells | | | | infiltrate with | |
| | in | | | | predominantly | |
| | papillary | | | | mononuclear cells. | |
| | dermis | | | | Macrophages: NS | |
| | | | | | Polymorphonuclear | |
| | | | | | cells: NS | |
| Spike 3 | Negative | Positive on | Positive on | 2/1 | Mild perivascular, | 8 mm |
| (2 doses of | | perivascular, | perivascular, | | periadnexal and | 26 COI |
| Sinopharm | | periadnexal | periadnexal | | perineural | |
| vaccine) | | and | and | | inflammatory | |
| | | perineural | perineural | | reaction pattern in | |
| | | mature- | mature- | | papillary dermis. | |
| | | looking T- | looking T- | | Inflammatory | |
| | | cells | cells | | infiltrate with | |
| | | | | | predominantly | |
| | | | | | mononuclear cells. | |
| | | | | | Macrophages: NS | |
| | | | | | Polymorphonuclear | |
| | | | | | cells: NS | |
| Spike 4 | Positive | Positive on | Positive on | 3/1 | Mild perivascular | 6 mm |
| (COVID + | on | perivascular | perivascular | | and periadnexal | 63 COI |
| 2 doses of | isolated | and | and | | inflammatory | |
| Sputnik V | mature- | periadnexal | periadnexal | | reaction pattern in | |
| vaccine) | looking | mature- | mature- | | papillary dermis. | |
| | B-cells | looking T- | looking T- | | Inflammatory | |
| | in | cells | cells | | infiltrate with | |
| | papillary | | | | predominantly | |
| | dermis | | | | mononuclear cells. | |
| | | | | | Macrophages: NS | |
| | | | | | Polymorphonuclear | |
| | | | | | cells: NS | |
| Spike 5 | Negative | Positive on | Positive on | 3/1 | Mild perivascular | 5 mm |
| (COVID + | | perivascular | perivascular | | and periadnexal | 209 |
| 2 doses of | | and | and | | inflammatory | COI |
| Sinopharm | | periadnexal | periadnexal | | reaction pattern in | |
| vaccine) | | mature- | mature- | | papillary dermis. | |
| | | looking T- | looking T- | | Inflammatory | |
| | | cells | cells | | infiltrate with | |
| | | | | | predominantly | |
| | | | | | mononuclear cells. | |
| | | | | | Macrophages: NS | |
| | | | | | Polymorphonuclear | |
| | | | | | cells: NS | |
| Spike 6 | Negative | Positive on | Positive on | 2/1 | Mild perivascular | 3 mm |
| (2 doses of | | perivascular | perivascular | | inflammatory | 123 |
| Sinopharm | | mature- | mature- | | reaction pattern in | COI |
| vaccine) | | looking T- | looking T- | | papillary dermis. | |
| | | cells | cells | | Inflammatory | |
| | | | | | infiltrate with | |
| | | | | | predominantly | |
| | | | | | mononuclear cells. | |
| | | | | | Macrophages: NS | |
| | | | | | Polymorphonuclear | |
| | | | | | cells: NS | |

Immunohistochemistry results of CD4+ T-cells, CD8+ T-cells and CD20+ B-cells staining on biopsies samples following spike or vehicle i.d. administration. NS: Not significant. mm: induration and/or erythema diameter in millimeters; IgG: anti-S protein serum IgG levels.

1. SARS-CoV-2-derived antigen for use in a method for determining whether a cellular and/or humoral immune response has occurred in subjects who have been infected with- or vaccinated against SARS-CoV-2 which comprises:
a. Determining the presence of a localized skin immune reaction comprising a wheal skin induration and/or erythema at or around the administration site once the SARS-CoV-2-derived antigen has been administered intradermally,
b. Wherein the presence of a localized skin immune reaction comprising a wheal skin induration and/or erythema at or around the administration site is an indication that the subjects have developed a cellular and/or humoral immune response against the SARS-CoV-2.

2. SARS-CoV-2-derived antigen for use, according to claim 1, wherein the presence of a localized skin immune reaction comprising wheal, skin induration and/or erythema at or around the administration site with a diameter longer than a pre-established threshold value, is an indication that the subjects have developed cellular and/or humoral immune response against SARS-CoV-2.

3. SARS-CoV-2-derived antigen for use, according to any of the claims 1 or 2, wherein the presence of a skin immune reaction comprising wheal, skin induration and/or erythema at or around the administration site with a diameter of its major axis, longer than a pre-established threshold value is an indication that the subjects have developed a cellular and/or humoral immune response against SARS-CoV-2.

4. SARS-CoV-2-derived antigen for use, according to any of the previous claims, wherein the presence of a skin immune reaction comprising skin induration and/or erythema at or around the administration site with a diameter of its major axis of a least 4.0 mm, is an indication that the subjects have developed a cellular and/or humoral immune response against the SARS-CoV-2.

5. SARS-CoV-2-derived antigen for use, according to any of the previous claims, further comprising assessing the presence or amount of infiltrating immunological cells and/or related markers in a biological sample obtained from the area of wheal, skin induration and/or erythema, wherein the presence of an increased amount of infiltrating immunological cells and/or their related markers with respect to a pre-established threshold value, is an indication that the subjects have developed cellular and/or humoral immune response against the SARS-CoV-2.

6. SARS-CoV-2-derived antigen for use, according to any of the previous claims, further comprising assessing the presence or amount of infiltrating lymphocytes, other mononuclear cells, polymorphonuclear cells, mast cells and/or related markers in a biological sample obtained from the area of wheal, induration and/or erythema, wherein the presence of an increased amount of infiltrating lymphocytes, other mononuclear cells, polymorphonuclear cells, mast cells and/or related markers, with respect to a pre-established threshold value, is an indication that the subjects have developed cellular and/or humoral immune response against the SARS-CoV-2.

7. SARS-CoV-2-derived antigen for use, according to any of the previous claims, further comprising assessing the presence or amount of infiltrating T cells, other mononuclear cells, polymorphonuclear cells and/or related markers in a biological sample obtained from the area of induration and/or erythema, wherein the presence of an increased amount of infiltrating T cells, other mononuclear cells, polymorphonuclear cells and/or related markers, with respect to a pre-established threshold value, is an indication that the subjects have developed cellular immune response against the SARS-CoV-2.

8. SARS-CoV-2-derived antigen for use, according to any of the previous claims, which further comprises assessing CD4⁺/CD8⁺ T cells ratio among the infiltrating cells in a biological sample obtained from the area of induration and/or erythema wherein the identification of a CD4⁺/CD8⁺ T-cells ratio different than a pre-established threshold ratio value, is an indication that the subjects have developed cellular immune response against the SARS-CoV-2.

9. SARS-CoV-2-derived antigen for use, according to any of the previous claims, which further comprises assessing CD4⁺/CD8⁺ T cells ratio among the infiltrating cells in a biological sample obtained from the area of induration and/or erythema, wherein the identification of a CD4⁺/CD8⁺ T cells ratio different than 1/1 is an indication that the subjects have developed cellular immune response against the SARS-CoV-2.

10. SARS-CoV-2-derived antigen for use, according to any of the previous claims, which further comprises assessing the presence or amount of infiltrating B cells and/or related markers in a biological sample obtained from the area of induration and/or erythema, wherein the presence of an increased amount of infiltrating B cells and/or their related markers with respect to a pre-established threshold value, is an indication that the subjects have developed humoral immune response against the SARS-CoV-2.

11. SARS-CoV-2-derived antigen for use, according to any of the previous claims, which further comprises assessing the presence or amount of infiltrating innate mononuclear cells, polymorphonuclear cells and/or related markers in a biological sample obtained from the area of induration and/or erythema, wherein the presence of an increased amount of infiltrating innate mononuclear cells, polymorphonuclear cells and/or their related markers with respect to a pre-established threshold value, is an indication that the subjects have developed cellular innate immune response against the SARS-CoV-2.

12. SARS-CoV-2-derived antigen for use, according to any of the previous claims, which further comprises assessing the presence or amount of infiltrating eosynophils, basophils, mast cells and/or related markers in a biological sample obtained from the area of wheal, induration and/or erythema, wherein the presence of an increased amount of infiltrating eosynophils, basophils, mast cells and/or their related markers with respect to a pre-established threshold value, is an indication that the subjects have developed IgE-mediated immune response against the SARS-CoV-2.

13. SARS-CoV-2-derived antigen for use, according to any of the previous claims, which further comprises performing an image examination to assess the characteristics of the dermis and subcutaneous cell tissues in the area at or around the antigen administration, wherein the identification of a different imaging pattern with respect to a pre-established imaging pattern, is an indication that the subjects have developed cellular and/or humoral immune response against the SARS-CoV-2.

14. SARS-CoV-2-derived antigen for use, according to any of the previous claims, which further comprises performing an image examination to assess the thickness of the dermis and subcutaneous cell tissues in the area at or around the antigen administration, wherein the identification of a thickness value higher than a pre-established threshold thickness value, is an indication that the subjects have developed cellular and/or humoral immune response against the SARS-CoV-2.

15. SARS-CoV-2-derived antigen for use, according to any of the previous claims, which further comprises performing an image examination to assess the thickness of the dermis and subcutaneous cell tissues in the area at or around the antigen administration, wherein the identification of a thickness value of at least twice a pre-established threshold value, preferably a thickness value of at least 2 mm, is an indication that the subjects have developed cellular and/or humoral immune response against the SARS-CoV-2.

16. SARS-CoV-2-derived antigen for use, according to any of the previous claims, which further comprises determining whether an IgE-mediated immune response has occurred in subjects who have been infected with- or vaccinated against SARS-CoV-2 by determining the presence of a wheal in the area at or around where the SARS-CoV-2-derived antigen has been administered intradermally, wherein the identification of a wheal between 10-30 minutes following the antigen administration is an indication that the subjects have developed IgE-mediated immune response against the SARS-CoV-2 and the vaccination of these subjects is potentially contraindicated.

17. SARS-CoV-2-derived antigen for use, according to any of the previous claims, in a method for recommending whether a subject should be vaccinated against SARS-CoV-2 by determining the presence of a wheal in the area at or around once the SARS-CoV-2-derived antigen has been administered intradermally, wherein the presence of a wheal in the area at or around the administration site between 10-30 minutes following the antigen administration is an indication that the subjects have developed IgE-mediated immune response against the SARS-CoV-2 infection and the vaccination of these subjects is potentially contraindicated.

18. SARS-CoV-2-derived antigen for use, according to any of the previous claims, in a method for determining whether cellular immune response has occurred in subjects who have been infected with- or vaccinated against SARS-CoV-2 by determining the presence of a skin induration and/or erythema at or around once the SARS-CoV-2-derived antigen has been administered intradermally, wherein the presence of a skin induration and/or erythema between 18-72 hours following the antigen administration is an indication that the subjects have developed cell-mediated immune response against the SARS-CoV-2.

19. SARS-CoV-2-derived antigen for use, according to any of the previous claims, in a method for determining whether IgG and/or IgM-mediated immune response has occurred in subjects who have been infected with- or vaccinated against SARS-CoV-2 by determining the presence of a skin induration and/or erythema at or around once the SARS-CoV-2-derived antigen has been administered intradermally, wherein the presence of a skin induration and/or erythema between 4-24 hours following the antigen administration is an indication that the subjects have developed IgG and/or IgM-mediated immune response against the SARS-CoV-2.

20. SARS-CoV-2-derived antigen for use, according to any of the previous claims, wherein the antigen is a molecule derived from SARS-CoV-2 which is able to elicit an immune response in the host, comprising: Structural proteins selected from Spike (S), Nucleocapsid (N), Membrane (M) or Envelope (E) protein or peptides derived from them; inactivated or attenuated viral particles; isolated genetic material or virus-like particles.

21. SARS-CoV-2-derived antigen for use, according to any of the previous claims, wherein the antigen is included in a pharmaceutical composition which optionally comprises pharmaceutically acceptable excipients and/or carriers.

22. SARS-CoV-2-derived antigen for use, according to any of the previous claims, in a computer-implemented method which comprises:
d. Receiving by a computer program the variables assessed according to any of the previous claims, once the SARS-CoV-2-derived antigen has been administered intradermally; preferably by means of an image taken from the skin.
e. Process the values according to step a) for finding a statistically significant variation or deviation
f. Providing a result by the computer system regarding the development of immune response based on the information entered according to the step a) and a pre-established threshold value already stored in the computer, wherein this result is communicated by the computer system to the subject and/or to the authorized health personnel and/or the health system.

23. A data processing apparatus, device or system comprising means for carrying out the method of claim 22.

24. Computer program product configured for carrying out the method of claim 22.

25. A kit which comprises:
a. A medical device for administering intradermally a SARS-CoV-2-derived antigen, and
b. Instructions for determining the presence of a wheal, skin induration and/or erythema at or around the administration site.

26. A kit, according to claim 25, which comprises:
a. A medical device for administering intradermally a SARS-CoV-2-derived antigen, and
b. Instructions for determining the presence of a wheal at or around the administration site between 10-30 minutes following the antigen administration.

27. A kit, according to claim 25, which comprises:
a. A medical device for administering intradermally a SARS-CoV-2-derived antigen, and
b. Instructions for determining the diameter of the major axis of the skin induration and/or erythema at or around formed between 18-72 or 4-24 hours following the antigen administration.
